(19) United States
(12) Patent Application Publication
(10) Pub. No.: US 2022/0309651 A1
(43) Pub. Date: Sep. 29, 2022

(54) METHOD, DEVICE, AND STORAGE MEDIUM FOR SEMI-SUPERVISED LEARNING FOR BONE MINERAL DENSITY ESTIMATION IN HIP X-RAY IMAGES

(71) Applicant: Ping An Technology (Shenzhen) Co., Ltd., Shenzhen (CN)

(72) Inventors: Kang ZHENG, Bethesda, MD (US); Shun MIAO, Bethesda, MD (US); Yirui WANG, Bethesda, MD (US); Xiaoyun ZHOU, Bethesda, MD (US); Le LU, Bethesda, MD (US)

(21) Appl. No.: 17/483,357

(22) Filed: Sep. 23, 2021

Related U.S. Application Data
(60) Provisional application No. 63/165,223, filed on Mar. 24, 2021.

(51) Int. Cl. G06T 7/00; G06T 7/11; G06N 3/08; G06N 3/04; A61B 6/00

(52) U.S. Cl. CPC G06T 7/0012; G06T 7/11; G06N 3/08; G06N 3/04; A61B 6/505; G06T 2207/30008; G06T 2207/20132; G06T 2207/20084; G06T 2207/20081; G06T 2207/10116

(57) ABSTRACT

A method for estimating bone mineral density (BMD) includes obtaining an image and cropping one or more regions-of-interest (ROIs) in the image, taking the one or more ROIs as input to a network model for estimating BMDs, training the network model on the labeled one or more ROIs with one or more loss functions to obtain a pre-trained model in a supervised pre-training stage, and fine-tuning the pre-trained model on a first plurality of data representing the labeled one or more ROIs and a second plurality of data representing unlabeled region to determine a fine-tuned network model for estimating BMDs in a semi-supervised self-training stage. The one or more loss functions includes a specific adaptive triplet loss (ATL) configured to encourage distances between one or more feature embedding vectors correlated to differences among the BMDs.

### CROSS-REFERENCE TO RELATED APPLICATION

This application claims the priority of U. S. Provisional Patent Application No. 63/165,223, filed on Mar. 24, 2021, the entire content of which is incorporated herein by reference.

### FIELD OF THE TECHNOLOGY

This application relates to the field of bone mineral density (BMD) estimation and, more particularly, relates to method, electronic device, and computer program product for estimating BMD from plain film hip X-ray images for osteoporosis screening.

### BACKGROUND OF THE DISCLOSURE

Osteoporosis is a common skeletal disorder characterized by decreased bone mineral density (BMD) and bone strength deterioration, leading to an increased risk of fragility fracture. All types of fragility fractures affect the elderly with multiple morbidities, reduced life quality, increased dependence, and mortality. A fracture risk assessment tool, FRAX has been clinically relied on for assessing bone fracture risks by integrating clinical risk factors and BMD. While some clinical risk factors such as age, gender, and body mass index (BMI) can be obtained from electronic medical records, the current gold standard to measure BMD is dual-energy X-ray absorptiometry (DEXA). However, due to the limited availability of DEXA devices, especially in developing countries, osteoporosis is often under-diagnosed and under-treated. Other methods aiming to use imaging obtained from other indications such as CT scans, and particularly high radiation dose of CT scans require longer acquisition time and higher costs, etc. Therefore, alternative lower-cost BMD evaluation protocols and methods using more accessible medical imaging examinations, e.g., X-ray plain films, can be a more accessible and lower-cost imaging tool for osteoporosis screening.

### SUMMARY

One aspect of the present disclosure provides a method for estimating bone mineral density (BMD). The method includes obtaining an image and cropping one or more regions-of-interest (ROIs) in the image, taking the one or more ROIs as input to a network model for estimating BMDs, training the network model on the labeled one or more ROIs with one or more loss functions to obtain a pre-trained model in a supervised pre-training stage, and fine-tuning the pre-trained model on a first plurality of data representing the labeled one or more ROIs and a second plurality of data representing unlabeled region to determine a fine-tuned network model for estimating BMDs in a semi-supervised self-training stage. The one or more loss functions includes a specific adaptive triplet loss (ATL) configured to encourage distances between one or more feature embedding vectors correlated to differences among the BMDs.

Another aspect of the present disclosure provides an electronic device for estimating bone mineral density (BMD). The electronic device includes a memory for storing a computer program and a processor coupled to the memory. When the computer program is executed, the computer program causes the processor to obtain an image and crop one or more regions-of-interest (ROIs) in the image, take the one or more ROIs as input to a network model for estimating BMDs, train the network model on the labeled one or more ROIs with one or more loss functions to obtain a pre-trained model in a supervised pre-training stage, and fine-tune the pre-trained model on a first plurality of data representing the labeled one or more ROIs and a second plurality of data representing unlabeled region to determine a fine-tuned network model for estimating BMDs in a semi-supervised self-training stage. The one or more loss functions includes a specific adaptive triplet loss (ATL) configured to encourage distances between one or more feature embedding vectors correlated to differences among the BMDs.

Another aspect of the present disclosure provides a computer program product for estimating bone mineral density (BMD). The computer program product includes a non-transitory computer-readable storage medium and program instructions. When executed, the program instructions cause a computer to obtain an image and crop one or more regions-of-interest (ROIs) in the image, take the one or more ROIs as input to a network model for estimating BMDs, train the network model on the labeled one or more ROIs with one or more loss functions to obtain a pre-trained model in a supervised pre-training stage, and fine-tune the pre-trained model on a first plurality of data representing the labeled one or more ROIs and a second plurality of data representing unlabeled region to determine a fine-tuned network model for estimating BMDs in a semi-supervised self-training stage. The one or more loss functions includes a specific adaptive triplet loss (ATL) configured to encourage distances between one or more feature embedding vectors correlated to differences among the BMDs.

Other aspects of the present disclosure can be understood by those skilled in the art in light of the description, the claims, and the drawings of the present disclosure.

### BRIEF DESCRIPTION OF THE DRAWINGS

FIG. 1 illustrates an example framework for a supervised pre-training according to various embodiments of the present disclosure.

FIG. 2 illustrates an example framework for a semi-supervised self-training stage according to various embodiments of the present disclosure.

FIG. 3 illustrates a flowchart of a method for training a model for estimating BMD on data representing a hip X-ray image according to various embodiments of the present disclosure.

FIG. 4 illustrates a flowchart of a method for training a model on the feature vectors of the ROI image using a mean square error (MSE) loss and a novel adaptive triplet loss (ATL) according to various embodiments of the present disclosure.

FIG. 5 illustrates an anchor sample, a near sample, and a far sample during an embedding learning for determining the novel ATL according to various embodiments of the present disclosure.

FIG. 6 illustrates a flowchart of a method for self-training the network model according to various embodiments of the present disclosure.

FIG. 7 illustrates errors occurred in predicted BMDs against the GT BMDs during the semi-supervised self-training according to various embodiments of the present disclosure.

FIG. 8 illustrates a structural diagram of an exemplary electronic device for performing the method for estimating BMDs using hip X-rays consistent with various embodiments of the present disclosure.

### DETAILED DESCRIPTION

The following describes the technical solutions in the embodiments of the present invention with reference to the accompanying drawings. Wherever possible, the same reference numbers will be used throughout the drawings to refer to the same or like parts. Apparently, the described embodiments are merely some but not all the embodiments of the present invention. Other embodiments obtained by a person skilled in the art based on the embodiments of the present invention without creative efforts shall fall within the protection scope of the present disclosure. Certain terms used in this disclosure are first explained in the followings.

Various embodiments provide method, electronic device, and computer program product of a method for estimating BMD from plain film hip X-ray images for osteoporosis screening. The various embodiments are based on the assumption that hip X-ray images contain sufficient information on visual cues for BMD estimation.

As used herein, the term “hip X-ray” refer to X-ray imaging results and/or X-ray examinations, that can help to detect bone cysts, tumors, infection of the hip joint, or other diseases in the bones of the hips, etc.

In some embodiments, a convolutional neural network (CNN) architecture is implemented for regressing BMD from hip X-ray images. For example, paired hip X-ray image and DEXA measured BMD are collected as labeled data for supervised regression learning. In some embodiments, the hip X-ray images and DEXA measured BMD are taken within six months apart. However, it can be difficult to obtain a large amount of hip X-ray images paired with DEXA measured BMDs.

A semi-supervised learning method may be implemented to exploit the usefulness of large-scale hip X-ray images without ground-truth BMDs. This method may make image collection easier than paring hip X-ray images with DEXA measured BMDs. Due to the continuity of BMD values, the model can be formulated as a regression model. In some embodiments, to improve regression accuracy, a novel adaptive triplet loss (ATL) method may be implemented such that the model can better distinguish samples with dissimilar BMDs in a feature space.

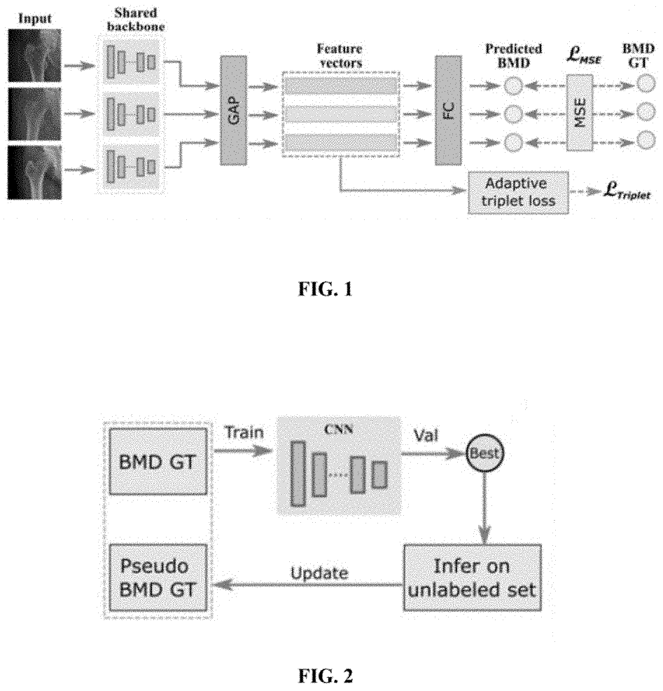

According to the embodiments of the present disclosure, training a model for estimating BMDs includes a supervised pre-training stage and a semi-supervised self-training stage. FIG. 1 illustrates a framework for a supervised pre-training, and FIG. 2 illustrates a framework for a semi-supervised self-training stage. The method for estimating BMDs includes two stages. During the first stage, a supervised pre-training is conducted to obtain a pre-trained network model. The obtained pre-trained model is subsequently used for self-training during the semi-supervised self-training stage.

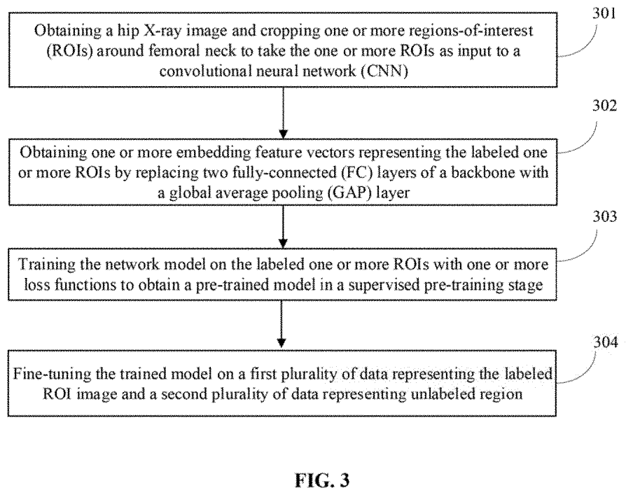

FIG. 3 illustrates a flowchart of a method for training a model for estimating BMD on data representing a hip X-ray image.

As shown in FIG. 3, in the supervised pre-training stage, a model may be trained on labeled images using a Mean Square Error (MSE) loss and a novel ATL. The novel ATL encourages distances between feature embeddings of samples correlated to their BMD difference.

In the self-training stage, the model may be fine-tuned on labeled data and pseudo-labeled data. The pseudo labels may be updated when the model achieves higher performance on the validation set.

Step 301: Obtaining a hip X-ray image and cropping one or more regions-of-interest (ROIs) around femoral neck to take the one or more ROIs as input to a convolutional neural network (CNN).

As shown in FIG. 3, in the supervised pre-training stage, in Step 301, a hip X-ray image may be obtained. For example, 1,090 hip X-ray images may be collected with associated DEXA-measured BMD values from 819 patients. The X-ray images may be taken within six months of the BMD measurement. The X-ray images may be split into training, validation, and test sets of 440 images, 150 images, and 500 images, respectively, based on patient identities. The hip X-ray image may be then cropped for a region-of-interest (ROI) around femoral neck. As such, the cropped ROI may be used as an input to the CNN. In one exemplary implementation, the ROIs may be resized to 512×512 pixels as model input. In some embodiments, to extract hip ROI images around the femoral neck, an automated ROI localization model may be trained with the deep adaptive graph (DAG) network using about 100 images with manually annotated anatomical landmarks. Random affine transformations, color jittering, and horizontal flipping may also be applied to resized ROI during training.

Step 302: Obtaining one or more embedding feature vectors representing the labeled one or more ROIs by replacing two fully-connected (FC) layers of a backbone with a global average pooling (GAP) layer.

In some embodiments, VGG-11 may be used as the backbone. In one example, VGG-11 may be adopted with batch normalization and squeeze-and-excitation (SE) layer as the backbone. The VGG-11 with batch normalization and the SE layer may outperform other VGG networks and ResNets. The last two fully-connected (FC) layers of VGG-11 may be replaced by a global average pooling (GAP) layer such that one or more embedding feature vectors may be obtained. In one example, the embedding feature vector includes a 512-dimensional embedding feature vector.

Step 303: Training the network model on the labeled one or more ROIs with one or more loss functions to obtain a pre-trained model in a supervised pre-training stage, the network model including the one or more embedding feature vectors.

After the one or more embedding feature vectors representing the labeled ROI image are obtained, one or more loss functions may be implemented to train a model on the one or more embedding feature vectors.

Step 304: Fine-tuning the trained model on a first plurality of data representing the labeled ROI image and a second plurality of data representing unlabeled region.

As shown in FIG. 3, in the self-training stage illustrated by step 304, the model may be fine-tuned on two groups of data. The two group of data includes a first plurality of data which represent the labeled ROI image and a second plurality of data which represent unlabeled region.

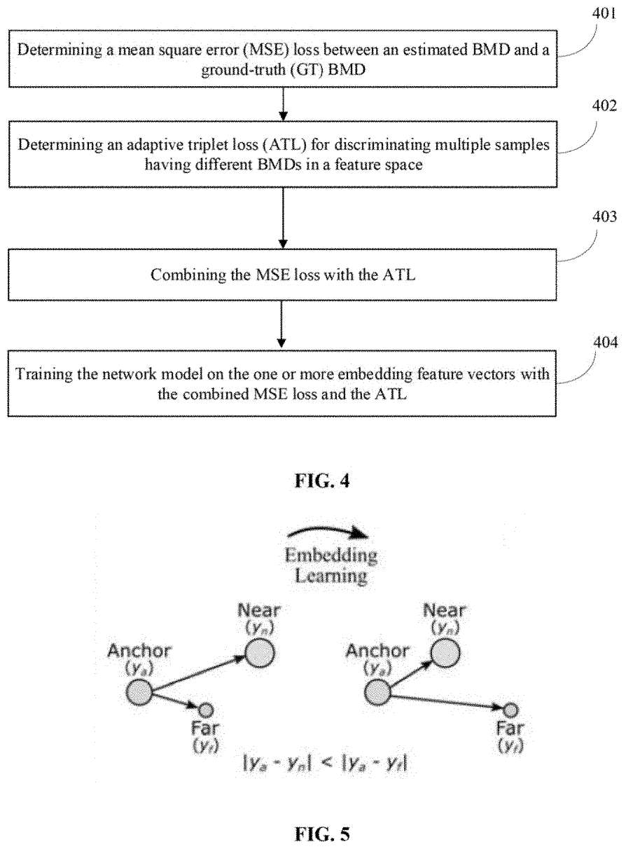

FIG. 4 illustrates a flowchart of a method for training a model representing the feature vectors of the ROI image using a mean square error (MSE) loss and a novel ATL.

Step 401: Determining a mean square error (MSE) loss between an estimated BMD and a ground-truth (GT) BMD.

After the one or more embedding feature vectors representing the labeled ROI image are obtained, a loss function may be implemented to train a model on the one or more embedding feature vectors. In some embodiments, in the supervised pre-training stage, the loss function used for training labeled ROI image may be, for example, mean square error (MSE) loss and an adaptive triplet loss.

As shown in FIG. 4, in step 401, a mean square error (MSE) loss may be first determined between a predicted BMD and a ground-truth (GT) BMD. The MSE loss can be determined by:

[symbol]

ₘₛₑ=( [symbol] − [symbol] )². (1)

where y′ denotes a predicted BMD, y denotes a GT BMD, Lₘₛₑ denotes the MSE loss.

According to formula (1) for determining the MSE loss, when a value of y′ approaches a value of y, the accuracy of the network model's regression can be maximized.

According to the embodiments of the present disclosure, BMD can be a continuous value, and embeddings of the hip ROIs can also be continuous in the feature space. In some embodiments, a distance between embeddings of two samples in the feature space can be correlated with their BMD discrepancy. Based on this characteristic, a novel ATL can be determined to discriminate samples with different BMDs in the feature space. FIG. 5 illustrates an anchor sample, a near sample, and a far sample during an embedding learning for determining the novel ATL according to the embodiments of the present disclosure.

Step 402: Determining an adaptive triplet loss (ATL) for discriminating multiple samples having different BMDs in a feature space.

As shown in FIG. 5, in one exemplary implementation, to determine the ATL, a first sample may be selected as anchor, a second sample having a BMD closer to that of the anchor is a near sample, and a third sample having a BMD further from that of the anchor than the second sample is a far sample. The relationship among the anchor sample, the near sample, and the far sample are determined by:

∥Fₐ−Fₙ∥₂²+m<∥Fₐ−F_{f}∥₂², (2)

where Fₐ, Fₙ, and F_{f} are embeddings of the anchor sample, the near sample, and the far sample, respectively, and m represents a margin that separates the near sample from the far sample. The margin accounts for the relative BMD differences between the near sample and the far sample. As such, the implementation of the ATL may encourage the distances between feature embeddings of samples correlated to their BMD difference.

Therefore, the ATL may be defined as:

[symbol]

=[∥Fₐ−Fₙ∥₂²−∥Fₐ−F_{f}∥₂²+αm]₊. (3)

where α is the adaptive coefficient based on the BMD differences, and can be defined by:

α=∥ [symbol] ₙ− [symbol] ₙ∥₂²−∥ [symbol] ₚ− [symbol] _{f}∥₂², (4)

where ya, yn, and yf are the GT BMD values of the anchor, near, and far samples, respectively.

Step 403: Combining the MSE loss with the ATL.

For network training, the MSE loss may be combined with the ATL. A weight may be considered for calculation. For example, the combined MSE loss and the ATL may be determined by:

[symbol]

= [symbol] ₘₛₑ+λ [symbol] ₜᵣᵢₚₗₑₜ, (5)

where λ represents a weight for the ATL. For example, λ can be 0.5 according to various embodiments of the present disclosure.

Step 404: Training the network model on the one or more embedding feature vectors with the combined MSE loss and the ATL.

The combined MSE loss and ATL may be used to train a network model on the one or more embedding feature vectors corresponding to the labeled ROI image. Accordingly, because of the implementation of the ATL, the trained model may learn more discriminative feature embeddings for image with different BMDs, thus improving the regression accuracy of the network.

When there are limited images coupled with GT BMDs, a network model can easily overfit the training data and yield poor performance on unseen test data. To overcome this barrier, a semi-supervised self-training algorithm can be implemented to leverage both labeled and unlabeled data. As such, a new semi-supervised self-training algorithm for boosting the BMD estimation accuracy can be implemented by exploiting unlabeled hip X-ray images. In one exemplary implementation, 1,090 hip X-ray images may be collected with associated DEXA-measured BMD values from 819 patients, and 8,219 unlabeled hip X-ray images may be collected.

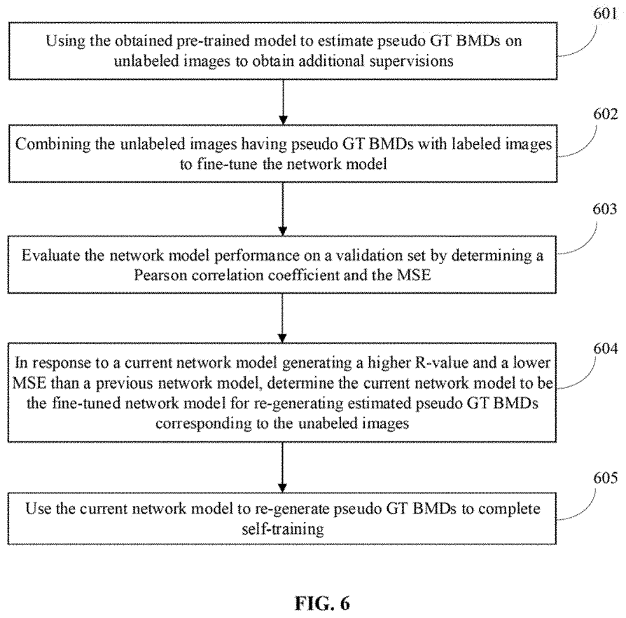

FIG. 2 illustrates an overview of a semi-supervised self-training stage, and FIG. 6 illustrates a flowchart of a method for self-training the network model.

Step 601: Using the obtained pre-trained model to estimate pseudo GT BMDs on unlabeled images to obtain additional supervisions.

The pre-trained model obtained from step 404 may be used to estimate pseudo GT BMDs on unlabeled images to obtain additional supervisions. The model may be fine-tuned on two groups of data. The two groups of data include a first plurality of data which represent the labeled ROI images and a second plurality of data which represent unlabeled regions. Accordingly, the trained model can be used to predict pseudo GT BMDs based on the unlabeled images to obtain additional supervisions, such that the unlabeled images with pseudo GT BMDs can be subsequently combined with labeled images to fine-tune the model.

Step 602: Combining the unlabeled images having pseudo GT BMDs with labeled images to fine-tune the network model.

The unlabeled images with pseudo GT BMDs may be combined with labeled images to fine-tune the network model. To improve the quality of estimated pseudo GT BMDs, a method for fine-tuning the network model is provided by the present disclosure. According to various embodiments of the present disclosure, a fine-tuned model can achieve higher performance on a validation set than the network model without fine-tuning. The fine-tuned model can also produce more accurate and more reliable pseudo GT BMDs for unlabeled images.

Step 603: Evaluate the network model performance on a validation set by determining a Pearson correlation coefficient and the MSE.

Two metrics for evaluation may be implemented for evaluating the proposed method and all compared methods, including Pearson correlation coefficient (R-value) and MSE or Root Mean Square Error (RMSE). In some embodiments, after each self-training stage, model performance on the validation set may be evaluated using the Pearson correlation coefficient and the MSE.

Step 604: In response to a current network model generating a higher R-value and a lower MSE than a previous network model, determine the current network model to be the fine-tuned network model for re-generating estimated pseudo GT BMDs corresponding to the unlabeled images.

If a fine-tuned model indeed achieves both higher correlation coefficient and lower MSE at the same time than a previous model, then the fine-tuned model may be used to re-generate pseudo GT BMDs for the unlabeled images during a self-training.

Step 605: Use the current network model to re-generate pseudo GT BMDs to complete self-training.

The fine-tuning process using the Pearson coefficient and the MSE as the evaluation factor may be repeated until a total self-training stage is achieved.

In one exemplary implementation, the semi-supervised self-training algorithm can be determined by the following process:
1: Initialize the best R-value \tilde{η}:=0 and MSE \hat{∈}:=
2: Initialize training epoch e:=0 and set total training epoch E
3: Initialize the model with pre-trained weights
4: while e<E do
5: Evaluate model performance R-value η and MSE ϵ on the validation set
6: If η>\tilde{η} and ϵ<\hat{∈} then
7: \tilde{η}:=η
8: \hat{∈}:=ϵ
9: Generate pseudo BMDs for unlabeled images
10: Fine-tune model on labeled images and unlabeled images with pseudo BMDs
11: e:=e+1

During a semi-supervised learning, optimization algorithm may be applied for training the learning models. For example, Adam optimizer with a learning rate of 10⁻⁴ and weight decay of 4×10⁻⁴ may be implemented to train the network on labeled images for 200 epochs. The learning rate may be decayed to 10⁻⁵ after 100 epochs. In one instance, the learning rate of 10⁻⁵ may maintain for another 100 epochs during the fine-tuning process. After each training and fine-tuning epoch, the network model may be evaluated on the validation set to select the highest Person correlation coefficient for testing. All models are implemented using PyTorch 1.7.1 and trained on a workstation with an Intel (R) Xeon (R) CPU, 128 G RAM, and a 12 G NVIDIA Titan V GPU, and a batch size may be set to 16.

Further, to regularize the network model and avoid being misled by inaccurate pseudo labels, each image may be augmented twice and consistency constraints may be employed between the features of each image and also between the predicted BMDs. In one exemplary implementation, consistency loss can be determined by:

[symbol]

_{c}=∥F₁−F₂∥₂²+∥ [symbol] ₁− [symbol] ₂∥₂². (6)
where assuming that I₁ and I₂ represent the two augmentations of a same image, respectively, F₁ and F₂ represent the features of the two augmentations I₁ and I₂ of the same image, and y₁ and y₂ represent the predicted BMDs corresponding to the two augmentations of the same image.

Based on the self-training network model provided in various embodiments, the total loss can be determined by:

[symbol]

= [symbol] ₘₛₑ+λ [symbol] ₜᵣᵢₚₗₑₜ+λ′ [symbol] _{c}, (7)

where λ′ represents a consistency loss weight. In various embodiments, λ^{t} may be set to 1.0.

According to the embodiments of the present disclosure, different backbones may affect the baseline performance without ATL or self-training. The compared backbones include VGG-11, VGG-13, VGG-16, ResNet-18, ResNet-34, and ResNet-50. As shown in Table 1 below, VGG-11 achieves the best R-value of 0.8520 and RMSE of 0.0831. The lower performance of other VGG networks and ResNets may be attributed to overfitting from more learnable parameters.

TABLE 1
Comparison of baseline methods using different backbones

| Backbone | VGG-11 | VGG-13 | VGG-16 | ResNet-18 | ResNet-34 | ResNet-50 |
|---|---|---|---|---|---|---|
| R-value | 0.8520 | 0.8501 | 0.8335 | 0.8398 | 0.8445 | 0.8448 |
| RMSE | 0.0831 | 0.0855 | 0.1158 | 0.0883 | 0.0946 | 0.1047 |

The present disclosure further provides comparison results between the semi-supervised self-training method according to the embodiments of the present disclosure and three existing semi-supervised learning (SSL) methods such as Π-model, temporal ensembling, and mean teacher. The Π-model is trained to encourage consistent network output between two augmentations of the same input image, the temporal ensembling produces pseudo labels via calculating the exponential moving average of predictions after every training epoch such that the pseudo labels may be then combined with labeled images to train the model, and the mean teacher uses an exponential moving average of model weights to produce pseudo labels for unlabeled images instead of directly ensembling predictions.

Regression MSE loss between predicted and GT BMDs can be used on labeled images for all SSL methods. All the SSL models may be fine-tuned from weights pre-trained on labeled images. As shown in Table 2, the semi-supervised self-training method according to the

TABLE 2
Comparison with semi-supervised learning methods.
(Temp. Ensemble: temporal ensembling)

| Method | Π-model | Temp. Ensemble | Mean Teacher | Proposed |
|---|---|---|---|---|
| R-value | 0.8637 | 0.8722 | 0.8600 | 0.8805 |
| RMSE | 0.0828 | 0.0832 | 0.0817 | 0.0758 |

embodiments of the present disclosure can achieve the best R-value of 0.8805 and RMSE of 0.0758. Π-model outperforms the baseline by enforcing output consistency as a regularization. While both temporal ensembling and mean teacher obtain improvements with the additional pseudo label supervision, averaging labels or weights can accumulate more errors over time. In contrast, the semi-supervised self-training method according to the embodiments of the present disclosure is more effective because it may only update pseudo labels when the model performs better on the validation set.

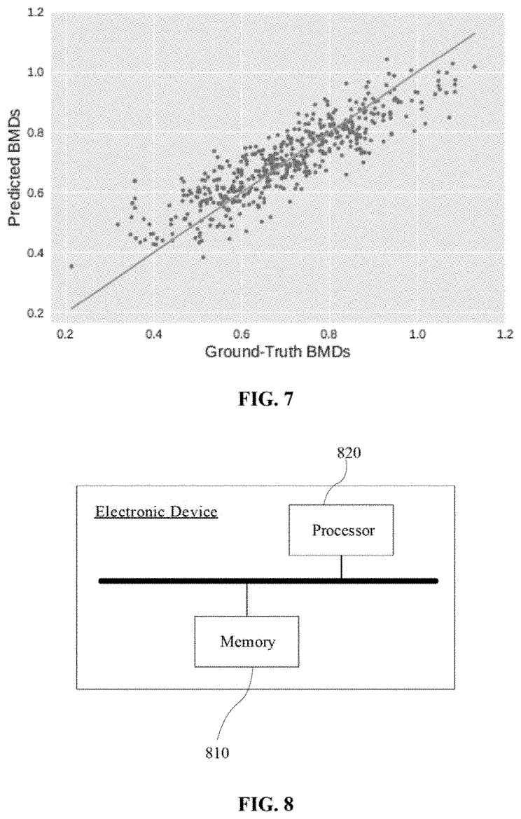

The predicted BMDs obtained according to the embodiments of the present disclosure are more evenly distributed in a medium range than end portions. FIG. 7 illustrates errors occurred in predicted BMDs against the GT BMDs during the semi-supervised self-training according to the embodiments of the present disclosure. As shown in FIG. 7, the semi-supervised self-training model may have a larger prediction error for lower or higher BMDs because lower or higher BMD cases are less common than the moderate BMD cases and the model tends to predict moderate values.

According to the embodiments of the present disclosure, the effectiveness of using ATL in training the network model is provided by comparing the model using the ATL with non-adaptive counterparts. To assess the importance of various components to the estimated BMDs, collected data may be grouped and different parameters may be applied to the data to evaluate the impact of the components on the BMDs. For example, some hyper-parameters may be varied while other hyper-parameters may remain among the groups of the data. In one exemplary implementation, the effectiveness of using ATL in training the model is compared with non-adaptive counterparts, at various preset margins. As shown below, Table 3 illustrates an ablation study of ATL.

TABLE 3
Ablation Study of Adaptive Triplet Loss (ATL)

| | |
|---|---|
| Triplet Loss | ATL |

| Margin | R-value | RMSE | R-value | RMSE |
|---|---|---|---|---|
| 0.1 | 0.8515 | 0.0917 | 0.8563 | 0.1013 |
| 0.3 | 0.8459 | 0.1000 | 0.8657 | 0.0814 |
| 0.5 | 0.8538 | 0.0866 | 0.8670 | 0.0806 |
| 0.7 | 0.8549 | 0.0823 | 0.8565 | 0.0836 |
| 1.0 | 0.8522 | 0.0829 | 0.8524 | 0.1215 |

As shown in Table 3, the non-adaptive counterpart deteriorates the model's regression accuracy. Therefore, the adaptive coefficient is necessary in achieving the network model's regression accuracy. Because BMD differences vary for different triplets, it may be unreasonable to use a fixed margin to uniformly separate samples with dissimilar BMDs. As shown in Table 3, the group of data using ATL can achieve higher R-values than the baseline regardless of the margin value (m). Specifically, when m=0.5, the data produces the best R-value of 0.8670 and RMSE of 0.0806.

In another exemplary implementation, one group of data use MSE loss only for fine-tuning the pre-trained model, and the other group of data use the combination of MSE loss and ATL loss for fine-tuning the pre-trained model. Table 4 illustrates an ablation study of adaptive triplet loss (ATL) and corresponding self-training algorithm.

TABLE 4
Ablation study of adaptive triplet loss
(ATL) and self-training algorithm.

| Method | R-value | RMSE |
|---|---|---|
| Baseline | 0.8520 | 0.0831 |
| Baseline + ATL | 0.8670 | 0.0806 |
| SSL | 0.8605 | 0.0809 |
| SSL + ATL | 0.8772 | 0.0767 |
| Proposed w/o Consistency | 0.8776 | 0.0761 |
| Proposed | 0.8805 | 0.0758 |

As shown in Table 4, in the first group of data, the R-value and RMSE are evaluated under the condition of having baseline components (denoted as “Baseline”) versus having baseline components and ATL loss (denoted as “Baseline+ATL”) in the pre-trained model; in the second group of data, the R-value and RMSE are evaluated under the condition of having the SSL loss (denoted as “SSL”) versus having the combination of SSL loss and the ATL loss (denoted as “SSL+ATL”); in the third group of data, the contribution of consistency loss in Equation 6 is illustrated, that is, the consistency of loss is removed during the self-training stage, and the R-value and RMSE are evaluated under the condition that the consistency has been removed in comparison with the condition that the consistency of loss has not been removed.

Moreover, as shown in Table 4, the implementation of a straightforward SSL strategy in the self-training stage can be effective in increasing the R-value and decreasing the RMSE value. In one example, the SSL increases the baseline R-value to 0.8605 and decreases the RMSE to 0.0809. Further, the pre-trained model using both the MSE loss and the ATL loss can further increase the R-value and decrease the RMSE. In addition, according to various embodiments of the present disclosure, while using pseudo labels of unlabeled images are effective in self-training stage, the R-values can be further increased and the RMSE can be further decreased when the pseudo labels are updated during fine-tuning. On the other hand, the consistency loss can regularize model training by encouraging consistent output and features. In some embodiments, the performance improvement of the R-value and RMSE becomes marginal in the situation where the pre-trained model does not use the consistency loss, and without the consistency loss, the model may be prone to overfitting to inaccurate pseudo labels and may deteriorate. For example, as shown in Table 4, the improvement becomes marginal from 0.8772 to 0.8776 in R-value without the consistency loss, even if pseudo labels are updated for multiple time during the fine-tuning process. Accordingly, when the self-training algorithm implements the adaptive coefficient ATL and consistency loss, a desirable R-value and RMSE can be achieved thus improving the regression accuracy of the network. For example, as shown in Table 4, in the data set with combined ATL and consistency loss applied to the self-training algorithm, a maximum R-value of 0.8805 and a minimum RMSE of 0.0758 can be achieved. Compared to the baseline, the R-value has been improved by 3.35% and the RMSE has been reduced by 8.78%.

Therefore, according to various embodiments of the present disclosure, a method of obtaining BMD from hip X-ray images instead of relying on the DEXA measurement is provided. A CNN may be employed to estimate BMDs from preprocessed hip ROIs. Further, to improve the regression accuracy of the network model, a novel ATL may be combined with MSE loss for training the network on hip X-ray images with paired ground-truth BMDs, thus providing feasibility of X-ray based BMD estimation and potential opportunistic osteoporosis screening with more accessibility and at reduced cost.

In various embodiments, the method for estimating BMDs provided by the present disclosure may be applied to one or more electronic devices.

In various embodiments, the electronic device is capable of automatically performing numerical calculation and/or information processing according to an instruction configured or stored in advance, and hardware of the electronic device can include, but is not limited to, a microprocessor, an Application Specific Integrated Circuit (ASIC), a Field-Programmable Gate Array (FPGA), a Digital Signal Processor (DSP), and an embedded device, etc. The electronic device can be any electronic product that can interact with users, such as a personal computer, a tablet computer, a smart phone, a desktop computer, a notebook, a palmtop computer, a personal digital assistant (PDA), a game machine, an interactive network television (IPTV), and smart wearable devices, etc. The electronic device can perform human-computer interaction with a user through a keyboard, a mouse, a remote controller, a touch panel, or a voice control device. The electronic device can also include a network device and/or a user device. The network device can include, but is not limited to, a cloud server, a single network server, a server group composed of a plurality of network servers, or a cloud computing system composed of a plurality of hosts or network servers. The electronic device can be in a network. The network can include, but is not limited to, the Internet, a wide region network, a metropolitan region network, a local region network, a virtual private network (VPN), and the like.

FIG. 8 illustrates a structural diagram of an exemplary electronic device for performing the method for estimating BMDs using hip X-rays consistent with various embodiments of the present disclosure.

Referring to FIG. 8, the exemplary electronic device includes a memory 810 storing a computer program, and a processor 820 coupled to the memory 810 and configured, when the computer program being executed, to perform the disclosed method for estimating BMDs using hip X-rays.

The memory 810 may include volatile memory such as random-access memory (RAM), and non-volatile memory such as flash memory, hard disk drive (HDD), or solid-state drive (SSD). The memory 810 may also include combinations of various above-described memories. The processor 820 may include a central processing unit (CPU), an embedded processor, a microcontroller, and a programmable device such as an application-specific integrated circuit (ASIC), a field programmable gate array (FPGA), and a programmable logic array (PLD), etc.

The present disclosure also provides a computer-readable storage medium storing a computer program. The computer program may be loaded to a computer or a processor of a programmable data processing device, such that the computer program is executed by the computer or the processor of the programmable data processing device to implement the disclosed method.

Various embodiments also provide a computer program product. The computer program product includes a non-transitory computer-readable storage medium and program instructions stored therein. The program instructions may be configured to be executable by a computer to cause the computer to implement the method for estimating BMDs using hip X-rays.

Although the principles and implementations of the present disclosure are described by using exemplary embodiments in the specification, the foregoing descriptions of the embodiments are only intended to help understand the method and core idea of the method of the present disclosure. Meanwhile, a person of ordinary skill in the art may make modifications to the specific implementations and application range according to the idea of the present disclosure. In conclusion, the content of the specification should not be construed as a limitation to the present disclosure.

What is claimed is:

1. A method for estimating bone mineral density (BMD), comprising:
obtaining an image and cropping one or more regions-of-interest (ROIs) in the image;
taking the one or more ROIs as input to a network model for estimating BMDs;
training the network model on the labeled one or more ROIs with one or more loss functions to obtain a pre-trained model in a supervised pre-training stage, the one or more loss functions including a specific adaptive triplet loss (ATL) configured to encourage distances between one or more feature embedding vectors correlated to differences among the BMDs; and
fine-tuning the pre-trained model on a first plurality of data representing the labeled one or more ROIs and a second plurality of data representing unlabeled region to determine a fine-tuned network model for estimating BMDs in a semi-supervised self-training stage.

2. The method according to claim 1, further comprising:
obtaining one or more embedding feature vectors representing the labeled one or more ROIs by replacing two fully-connected (FC) layers of a backbone with a global average pooling (GAP) layer.

3. The method according to claim 2, wherein the network model trained with the one or more loss functions.

4. The method according to claim 1, wherein the image is a hip X-ray image having visual cues for estimating BMDs, and the ROIs are cropped around a femoral neck of the hip.

5. The method according to claim 3, further comprising:
determining a mean square error (MSE) loss between an estimated BMD and a ground-truth (GT) BMD; and
determining an adaptive triplet loss (ATL) for discriminating multiple samples having different BMDs in a feature space.

6. The method according to claim 5, further comprising:
combining the MSE loss with the ATL; and
training the network model on the one or more embedding feature vectors with the combined MSE loss and the ATL.

7. The method according to claim 5, further comprising:
for each image, obtaining two augmentations and estimated BMDs corresponding to the two augmentations of each respective image, and determining a consistency loss;
combining the MSE loss, the ATL, and the consistency loss; and
training the network model on the one or more embedding feature vectors with the combined losses.

8. The method according to claim 1, further comprising:
estimating pseudo ground truth (GT) BMDs on unlabeled images with the obtained pre-trained model for additional supervision; and
combining the unlabeled images having pseudo GT BMDs with labeled one or more ROIs to fine-tune the pre-trained network model.

9. The method according to claim 8, further comprising:
estimating the fine-tuned network model on a validation set by determining a Pearson correlation coefficient (R-value) and the MSE.

10. The method according to claim 9, further comprising:
in response to a current network model generating a higher R-value and a lower MSE than a previous network model, determining the current network model to be the fine-tuned network model for re-generating estimated pseudo GT BMDs corresponding to the unlabeled images; and
re-generating pseudo GT BMDs using the fine-tuned network model to complete self-training.

11. The method according to claim 1, wherein the network is a convolutional neural network (CNN).

12. An electronic device for estimating bone mineral density (BMD), comprising:
a memory for storing a computer program; and
a processor coupled to the memory, when executed, the computer program causing the processor to:
obtain an image and crop one or more regions-of-interest (ROIs) in the image;
take the one or more ROIs as input to a network model for estimating BMDs;
train the network model on the labeled one or more ROIs with one or more loss functions to obtain a pre-trained model in a supervised pre-training stage, the one or more loss functions including a specific adaptive triplet loss (ATL) configured to encourage distances between one or more feature embedding vectors correlated to differences among the BMDs; and
fine-tune the pre-trained model on a first plurality of data representing the labeled one or more ROIs and a second plurality of data representing unlabeled region to determine a fine-tuned network model for estimating BMDs in a semi-supervised self-training stage.

13. The electronic device according to claim 12, wherein:
the network model is trained with the one or more loss functions; and
the processor is further configured to:
obtain one or more embedding feature vectors representing the labeled one or more ROIs by replacing two fully-connected (FC) layers of a backbone with a global average pooling (GAP) layer.

14. The electronic device according to claim 12, wherein the image is a hip X-ray image having visual cues for estimating BMDs, and the ROIs are cropped around a femoral neck of the hip.

15. The electronic device according to claim 13, wherein the processor is further configured to:
determine a mean square error (MSE) loss between an estimated BMD and a ground-truth (GT) BMD;
determine an adaptive triplet loss (ATL) for discriminating multiple samples with different BMDs in a feature space;
combine the MSE loss with the ATL; and
train the network model on the one or more embedding feature vectors with the combined MSE loss and the ATL.

16. The electronic device according to claim 12, wherein the processor is further configured to:
estimate pseudo ground truth (GT) BMDs on unlabeled images with the obtained pre-trained model for additional supervision; and
combine the unlabeled images having pseudo GT BMDs with labeled one or more ROIs to fine-tune the pre-trained network model.

17. The electronic device according to claim 16, wherein the processor is further configured to:
estimate the fine-tuned network model on a validation set by determining a Pearson correlation coefficient (R-value) and the MSE.

18. The electronic device according to claim 17, wherein the processor is further configured to:
in response to a current network model generating a higher R-value and a lower MSE than a previous network model, determine the current network model to be the fine-tuned network model for re-generating estimated pseudo GT BMDs corresponding to the unlabeled images; and
re-generate pseudo GT BMDs using the fine-tuned network model to complete self-training.

19. The electronic device according to claim 12, wherein the network is a convolutional neural network (CNN).

20. A computer program product for estimating bone mineral density (BMD), comprising:
a non-transitory computer-readable storage medium; and
program instructions, when executed, causing a computer to:
obtain an image and crop one or more regions-of-interest (ROIs) in the image;
take the one or more ROIs as input to a network model for estimating BMDs;
train the network model on the labeled one or more ROIs with one or more loss functions to obtain a pre-trained model in a supervised pre-training stage, the one or more loss functions including a specific adaptive triplet loss (ATL) configured to encourage distances between one or more feature embedding vectors correlated to differences among the BMDs; and
fine-tune the pre-trained model on a first plurality of data representing the labeled one or more ROIs and a second plurality of data representing unlabeled region to determine a fine-tuned network model for estimating BMDs in a semi-supervised self-training stage.
